(19) United States
(12) Patent Application Publication
(10) Pub. No.: US 2022/0151395 A1
(43) Pub. Date: May 19, 2022

(54) Smart Chair

(71) Applicant: Gregory Commercial Furniture Pty Limited, Eastern Creek (AU)

(72) Inventors: Scott Downes, Eastern Creek (AU); Amin Konjedkar, Eastern Creek (AU); Joel Murray, Eastern Creek (AU); Anthony Mankarios, Eastern Creek (AU); Greg Welsh, Eastern Creek (AU)

(21) Appl. No.: 17/199,334

(22) Filed: Mar. 11, 2021

(30) Foreign Application Priority Data
Apr. 24, 2020 (AU) 2020901303

(51) Int. Cl. A47C 31/12; A47C 7/62; A61B 5/00

(52) U.S. Cl. CPC A47C 31/126; A47C 7/62; A61B 2562/0247; A61B 5/4561; A61B 5/6891

(57) ABSTRACT

A smart chair and logging/warning assembly are disclosed. In some implementations, the smart chair includes a base arranged to receive a sitter, a sensor, and a transmitter. The sensor can be located within the base. Additionally, the sensor can take initial readings relating to the sitter on the chair for reference. Further, the sensor can take readings of the sitter while they are sitting and transmit information relating to the sitter's sitting to a computational device.

### FIELD

The present invention relates to a smart chair and logging/warning assembly.

The present invention has been developed primarily for use in office chairs. However, it will be appreciated that the invention is not limited to this particular field of use and is also suitable for other types of chairs and seats. For example, car seats, sofas, public seating, waiting room seating, baby chairs or otherwise as understood by the skilled addressee.

### BACKGROUND

Seats are well known and typically used directly support a sitter. As the sitter is often seated in the chair for prolonged periods of time, especially during work hours where the work requires sitting, the sitter can experience significant lower back pain, discomfort, increased risk of thrombosis and diseases associated with a sedentary life. This can present a workplace health and safety issue, reduce the efficiency and productivity of a workplace and burden the healthcare system.

In an attempt to reduce lower back pain experienced by the sitter, there have been numerous developments in seat cushions that seek to promote correct posture in the sitter. Specifically, known seat cushions have been designed to encourage the lower spine of the sitter to display a natural concave curvature known as lumbar lordosis. However, these known seat cushions do not cater well for ranging body shapes and sizes and therefore often fail to adequately promote correct posture, particularly in heavier sitters. Further, these known seat cushions often exert significant pressure on the thighs of the sitter restricting blood flow and causing discomfort, particularly in lighter sitters.

Further improvements in the design of seating cushions, such as those described in Australian Patent No. 2015349590, the contents of which are hereby incorporated by reference, have improved the above noted concerns, but are unable to eliminate them entirely.

Despite these and other developments in seating design, in situations where a sitter is sitting for extended periods, discomfort and health risks are a consideration.

### OBJECT OF INVENTION

It is the object of the present invention to overcome or substantially ameliorate one or more of the above disadvantages, or at least provide a useful alternative.

### SUMMARY OF INVENTION

In a first embodiment, there is disclosed herein a smart chair, including:
a base arranged to receive a sitter, a sensor and a transmitter;
wherein the sensor is located within the base;
wherein the sensor is arranged to take initial readings relating to a sitter on the chair for reference; and
wherein the sensor takes readings of a sitter while they are sitting and transmits information relating to the sitter's sitting to a computational device.

Preferably, the time a sitter sits on a chair is recorded.

Preferably, wherein the readings of a sitter include at least one of the sitter's weight, the sitter's weight distribution on the chair, the sitter's posture and the movement of the sitter on the smart chair.

Preferably, the computational device is arranged to analyse the information relating to a sitter's sitting and assigns a risk to the sitter based on the analysis.

Preferably, an active notification is sent to the sitter when the risk is assessed as too high.

An active notification is one that presents itself to the sitter without the sitter's input so that the sitter is presented with the warning at a set time.

Preferably, the notification is an alarm.

Preferably, the information related to a sitter's sitting is stored for future reference.

Preferably, the information related to a sitter's sitting is arranged to be sent to a third party.

In a second embodiment, there is disclosed herein a smart chair, including:
a back arranged to support a sitter, a sensor and a transmitter;
wherein the sensor is located within the back;
wherein the sensor is arranged to take initial readings relating to a sitter on the chair for reference; and
wherein the sensor takes readings of a sitter while they are sitting and transmits information relating to the sitter's sitting to a computational device.

In a third embodiment, there is disclosed herein an assembly for monitoring the sitting of a sitter, including a smart chair and a logging and/or warning system, wherein the smart chair includes at least one sensor and a low power transmitter connected to the at least one sensor arranged to transmit data related to the sitting of the sitter;

wherein the logging and/or warning system is arranged to analyse the data relating to the sitting of the sitter, assign a risk to the sitting of the sitter and actively notify the sitter if the risk is deemed too high.

Preferably, the time a sitter has been sitting contributes to the assigned risk.

In a fourth embodiment, there is disclosed herein a smart chair, including:

a base arranged to receive a sitter, a back arranged to support the sitter, a sensor and a transmitter;

wherein the sensor is located within at least one of the base and the back;

wherein the sensor is arranged to take initial readings relating to a sitter on the chair for reference; and
wherein the sensor takes readings of a sitter while they are sitting and transmits information relating to the sitter's sitting to a computational device

### BRIEF DESCRIPTION OF DRAWINGS

A preferred embodiment of the invention will be described hereinafter, by way of example only, with reference to the accompanying drawings, wherein:

FIG. 1 is a side sectional view of an embodiment of a smart chair according to the present invention;

FIG. 2 is a schematic view of sensors in the chair of FIG. 1;

FIG. 3 is a schematic view of a processing system used with the seat of FIG. 1;

FIG. 4 is a schematic view of a processing system used with the seat of FIG. 1;

FIG. 5 is a schematic view of a processing system used with the seat of FIG. 1;

FIG. 6 is a schematic view of a processing system used with the seat of FIG. 1.

### DESCRIPTION OF EMBODIMENTS

Broadly, the present invention relates to a smart chair 10 arrangement including at least one sensor 16 a lower power transmitter 18 and a logging/warning system 36. The sensor 16 is arranged to sense sitter data and sitting data relating to a sitter on the smart chair and transmit it to the logging and/or warning system 36. The logging and/or warning system 36 is arranged to calculate health risks to the sitter and actively notify the sitter of health risks when the risks for a sitter pass above a defined level.

The sensor 16 can comprise weight sensors arranged to calculate the weight of an object on the smart chair 10, location sensors arranged to identify the position of an object on the smart chair 10, pressure sensors arranged to identify the weight distribution of an object on the smart chair 10, scanning sensors arranged to identify the outline and or size of an object on the smart chair, Location sensors such as GPS sensors or otherwise, for example GLOSNASS sensors, as understood by the skilled addressee.

The sensor 16 may also be able to record the tilting and movement of a smart chair 10. The logging and/or warning system 26 is also able to record, track and compare data relating to multiple smart chairs at once.

In one embodiment, the low power transmitter 18 is a Bluetooth™ low energy module powered by a CR2032 battery. The skilled address will recognise that alternative low energy wireless transmitters and batteries can be used and fall within the scope of the present invention provided that they provide the ability to transfer data for extended periods of time in the range of years. To allow further data analysis related to a particular smart chair 10, each low power transmitter 18 can have a unique identifier that transmits with the sitter and sitting data.

The logging and or warning system is a computational system, such as a smart phone or other portable computational device, that can alert the sitter to health risks above a defined level through the use of notification systems such as alarms or otherwise. The sitter, or a third party such as an employer is able to review the sitter or sitting data for analysis and the presentation of data for analysis.

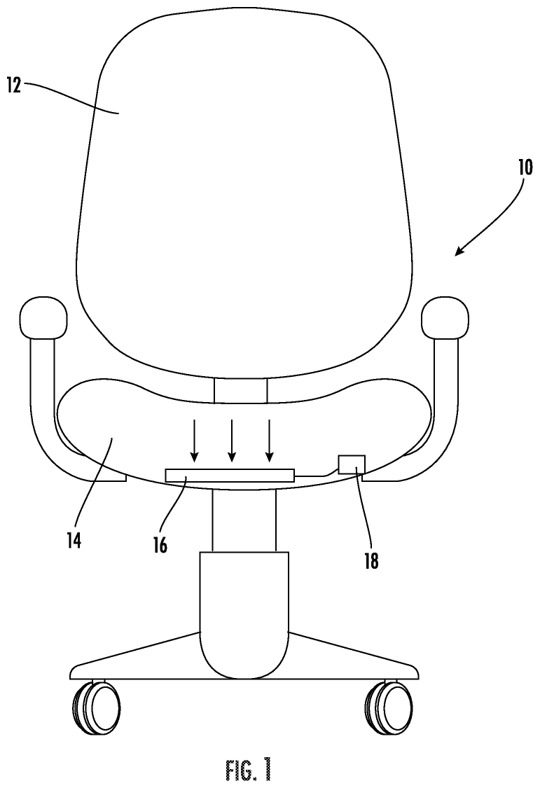

FIG. 1 shows an embodiment of a smart chair 10 including a chair back 12 and base 14. The skilled addressee will recognise that although the examples given in this specification relate to a smart chair with a back 12 and base 14 that the invention can equally apply to a smart chair with solely a base 14. It is within the scope of the present invention for the smart chair 10 to be fixed or movable, such as on rollers or otherwise. The smart chair 10 can also be a wheel chair or chair designed for people with reduced mobility.

In the base 14 of the smart chair 10 are located sensor 16 connected to lower power transmitter 18. The sensors take sitter data readings when a sitter first sits on the chair and transfers the data via low power transmitter 18. The sensor 16 also takes sitting data readings over time while the sitter is sitting and transfers the data via low power transmitter 18.

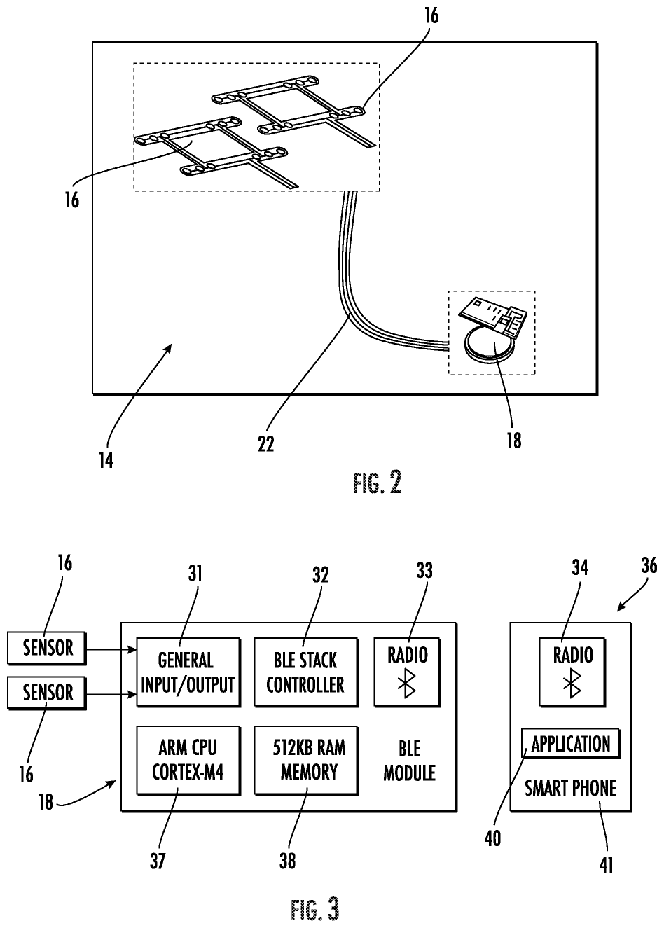

FIG. 2 illustrates a plurality of sensors 16 connected to low power transmitter 18. It is within the scope of the present invention for there to be a plurality of sensors 16 in the smart chair 10.

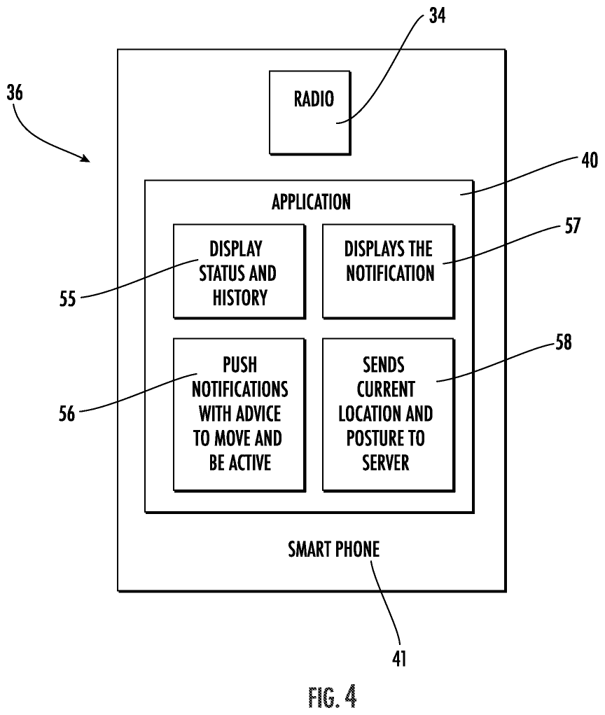

FIG. 3 illustrates the interaction between the sensor/s 16 with the low power transmitter 18 and the logging and/or warning system 36. The sensor/s 16 are arranged to transfer data relating to the sitter or sitting data to the low power transmitter 18.

Where the low power transmitter 18 is a Bluetooth™ transmitter it includes a processor 37, an input/output channel 31 connected to the sensor/s 16, RAM 38, a Bluetooth™ control stack 32 and a Bluetooth™ radio transmitter/receiver 33. The low power transmission unit 18 is arranged to transfer the sitter or sitting data to the Bluetooth™ radio transmitter/receiver 34 in the logging and/or warning system 36. In one embodiment, the logging and/or warning system is a smart phone with an onboard computing system and software applications 40 to analyse the sitter or sitting data. As noted above, the skilled addressee will recognise that the above features apply to broader wireless communication systems that operate over short distances.

FIG. 4 illustrates features of the logging and/or warning system 36. Sitter or sitting data is collected through Bluetooth™ radio transmitter/receiver 34 and processed by software application 40 on the logging and/or warning system 36. The software application uses the sitter data to build reference data describing a sitter. This data can include, weight, height, girth and posture. With the sitter data loaded as a reference the application 40 is able to process the data for the display of notifications 57. This can include visual notification, auditory notification, vibration notifications or otherwise. The application 40 stores and sends readings from the sensors 16 through application sensor module 58, the application 40 has identified a risk, notification module 56 sends a risk notification to the sitter and/or a third party such as an employer. The risk notification can be in the form of an alarm.

The application 40 stores the sitter and sitting readings in status and history module 55. This data can be used to assist a sitter in healthier sitting.

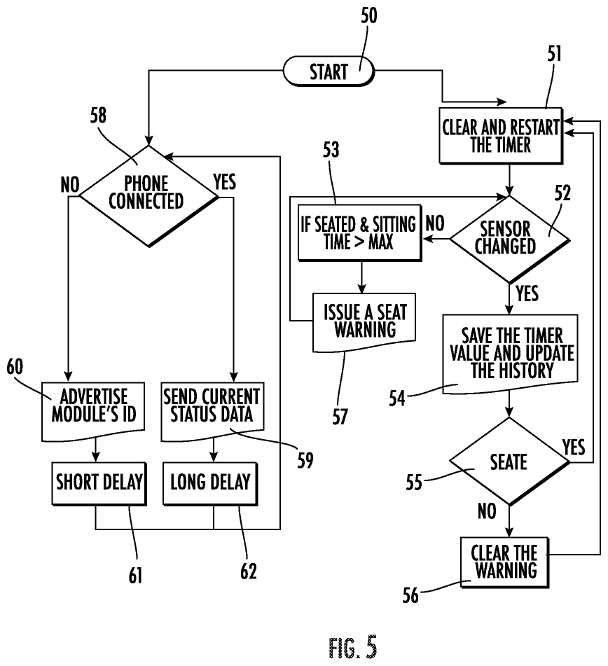

With reference to FIG. 5, a schematic of the operation of an embodiment of the application is shown. The logging and/or warning system 36 at phone testing step 58 tests for a connection between the low power transmitter 18 and the logging and/or warning system 36 where it is embodied on a smart phone. When no connection is detected the phone emits a identification signal at identification step 60. When a connection is detected phone status data is sent at status data step 59 to the application 40. This allows the application to identify that a sitter is in proximity to the smart chair.

With a sitter in proximity identified, the application initiation step 50 is triggered. From initiation step 50 a timer is started from a reset state at timer step 51. This provides a reference for the sitting data collected.

Sensor change step 52 detects if there is a change to the sensor 16 in the smart chair 10. This will provide data such as the weight of the sitter, the profile of their contact with the chair, weight distribution over the chair and movement of the sitter. If the connected phone is identified as a previous user the data for a sitter linked to the logging and\or warning system 36 can be referenced for comparative analysis.

If sensor change step 52 detects a change in the sensor indicating that a sitter has sat or risen from sitting, the duration since the resetting of the timer 51 is saved and recorded at recordal step 54.

If the sensor change step 52 detects no change to the sensor the sitting analysis step 53 calculates a danger risk based on at least the time a sitter has been sitting. If the calculation is over a set limit a warning alert is actively pushed to the sitter using an alarm or other active notification, such as a vibration, and the system goes back to sensor change step 52.

From recordal step 52, the logging and/or warning system 36, analyses from the sensor 16 readings and tests if the sitter is seated or has risen at sitting step 55. If the sitter is not sitting the warning alert is cancelled at cancellation step 56 and the system returns to timer step 51. If the sitter is identified as sitting at sitting step 55 no action is taken on the state of any active warning alerts and the system is returned to timer step 51.

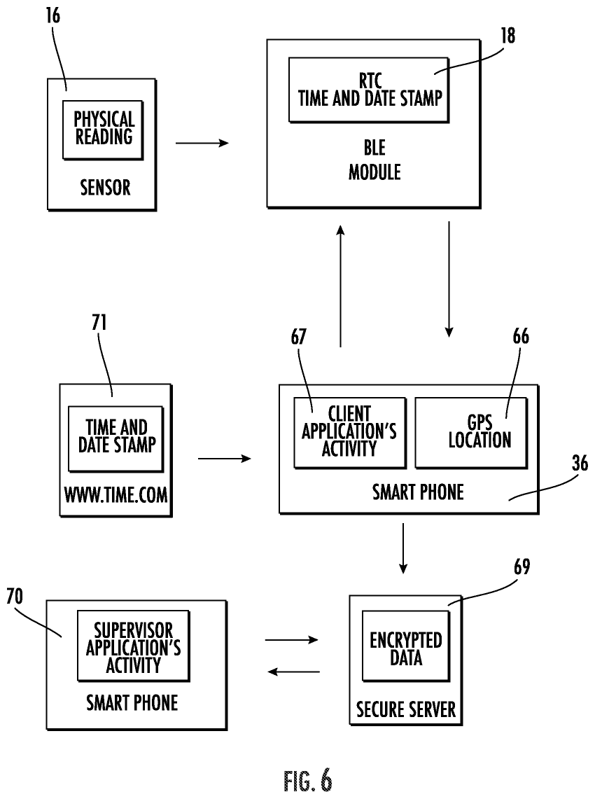

FIG. 6 shows the operation of the smart chair 16 with the low power transmitter 18 and logging and/or warning system 36. Sensor 16 sends data to the low power transmitter 18 through a physical connection between the two. Data is transferred between the low power transmitter 18 and the logging and/or warning system 36. A time and date stamp can be included with data transfers from or to the low power transmitter 18. The logging and/or warning system 36 may put a second date and time stamp 71. The logging and/or warning system 36 can include a GPS tracker 66 and a sitter activity log 67 of a sitter's historical interactions with the smart chair 10.

If the data collected by the logging and/or warning system 36 is to be sent to a third party, it is sent to a third parties computational device 70. In one embodiment, the data collected is encrypted.

As the logging and/or warning system 36 records individual identifications of a logging and/or warning system 36 used and the identify of a particular low power transmitter 18 the sitting patterns of individuals and groups can be tracked. These features can also be used to track and record the location of a smart chairs 10.

It is within the scope of the present invention for the sitter and sitting data recorded by a sensor 16 to be transferred to the logging and/or warning system 36 via a peer to peer network connecting a plurality of smart chairs 10 together. This allows data relating to a plurality of chairs to be extracted from a single chair.

Although the invention has been described with reference to a preferred embodiment, it will be appreciated by a person skilled in the art that the invention may be embodied in many other forms.

1. A smart chair, comprising:
a base arranged to receive a sitter;
a sensor; and
a transmitter,
wherein the sensor is located within the base;
wherein the sensor is arranged to:
take initial readings relating to the sitter on the smart chair for reference;
take readings of the sitter while they are sitting; and
transmit information relating to the sitter's sitting to a computational device.

2. The smart chair as claimed in claim 1, wherein a time the sitter sits on the smart chair is recorded.

3. The smart chair as claimed in claim 1, wherein the readings of the sitter include at least one of the sitter's weight, the sitter's weight distribution on the smart chair, the sitter's posture and the movement of the sitter on the smart chair.

4. The smart chair as claimed in claim 1, wherein the computational device is arranged to:
analyse the information relating to the sitter's sitting; and
assign a risk to the sitter based on the analysis.

5. The smart chair as claimed in claim 4, wherein the computational device is further arranged to send a notification to the sitter when the risk is assessed as exceeding a limit.

6. The smart chair as claimed in claim 5, wherein the notification is an alarm.

7. The smart chair as claimed in claim 1, wherein the information related to the sitter's sitting is stored for future reference.

8. The smart chair as claimed in claim 1, wherein the information related to the sitter's sitting is arranged to be sent to a third party.

9. A smart chair, comprising:
a back arranged to support a sitter;
a sensor; and
a transmitter,
wherein the sensor is located within the back; and
wherein the sensor is arranged to:
take initial readings relating to the sitter on the smart chair for reference; and
take readings of the sitter while they are sitting; and
transmit information relating to the sitter's sitting to a computational device.

10. A system that monitors sitting of a sitter, the system comprising:
a smart chair; and
a logging and/or warning system,
wherein the smart chair includes:
at least one sensor, and
a low power transmitter connected to the at least one sensor and arranged to transmit data related to the sitting of the sitter;
wherein the logging and/or warning system is arranged to:
analyse the data relating to the sitting of the sitter,
assign a risk to the sitting of the sitter, and
actively notify the sitter if the risk exceeds a limit.

11. The system as claimed in claim 10, wherein the time the sitter has been sitting contributes to the assigned risk.

12. A smart chair, comprising:
a base arranged to receive a sitter;
a back arranged to support the sitter;
a sensor; and
a transmitter,
wherein:
the sensor is located within at least one of the base and the back;
the sensor is arranged to take initial readings relating to the sitter on the smart chair for reference; and
the sensor is arranged to take readings of the sitter while they are sitting and to transmit information relating to the sitter's sitting to a computational device.
